(19) United States
(12) Patent Application Publication
(10) Pub. No.: US 2019/0336319 A1
(43) Pub. Date: Nov. 7, 2019

(54) PORTABLE FEMALE URINAL

(71) Applicant: MARTHA FALLIS, CLEARWATER, FL (US)

(72) Inventor: MARTHA FALLIS, CLEARWATER, FL (US)

(21) Appl. No.: 16/511,829

(22) Filed: Jul. 15, 2019

Related U.S. Application Data
(63) Continuation-in-part of application No. 14/919,691, filed on Oct. 21, 2015, now abandoned.
Application No. 14/919,691 is a continuation-in-part of application No. 13/998,056, filed on Sep. 26, 2013, now abandoned.
Application No. 13/998,056 is a continuation-in-part of application No. 13/742,926, filed on Jan. 16, 2013, now Pat. No. 8,603,056.

(51) Int. Cl. A61F 5/455; A61F 5/44

(52) U.S. Cl. CPC A61F 5/4556; A61F 5/4553; A61F 5/4408

(57) ABSTRACT

A collector has a bottom and an open top and a peripheral wall. The collector has an outlet opening. The bottom and the peripheral wall form a chamber. The chamber has a front and rear and sides and a peripheral edge adjacent to the open top. The peripheral edge is positionable inter-labially between the labia majora to cover a user's genitalia including the user's vulvar vestibule and urethral orifice/meatus, with the chamber front posterior to the user's clitoris and with the chamber rear overlying soft tissue in proximity to the labia majora and vagina. The collector is held in place during use solely by the fingers of the user adhesive free and insert free.

### RELATED APPLICATIONS

The present application is a continuation-in-part of U.S. patent application Ser. No. 14/919,691 filed Oct. 21, 2015 which is, in turn, a continuation-in-part of abandoned U.S. patent application Ser. No. 13/998,056 filed Sep. 26, 2013, which is a continuation-in-part of U.S. patent application Ser. No. 13/742,926 filed Jan. 16, 2013 which issued Dec. 10, 2013 as U.S. Pat. No. 8,603,056, the subject matter of which applications is incorporated herein by reference.

### BACKGROUND OF THE INVENTION

### Field of the Invention

The present invention relates to a portable female urinal and more particularly pertains to facilitating inter-labial positioning and urine collecting, the positioning and collecting being done in a sanitary, safe, discreet, convenient and economical manner.

### SUMMARY OF THE INVENTION

In view of the disadvantages inherent in the known types of urinals now present in the prior art such as U.S. Pat. No. 6,592,560 to Snyder, the present invention provides an improved portable female urinal. As such, the general purpose of the present invention, which will be described subsequently in greater detail, is to provide a new and improved portable female urinal and method which has all the advantages of the prior art and none of the disadvantages.

To attain this, the present invention essentially comprises a portable female urinal. The portable female urinal includes a collector having a bottom and an open top and a peripheral wall. The collector has an outlet opening. The bottom and the peripheral wall form a chamber. The chamber has a front and rear and sides and a peripheral edge adjacent to the open top. The position in an adult female: peripheral edge is positionable inter-labially between the labia majora to cover a user's genitalia including the user's clitoris and urethral orifice, with the chamber front overlying the user's pubic symphysis and clitoris and with the chamber rear overlying soft tissue anterior to the vaginal introitus and anus. The collector is held in place during use solely by the fingers of the user adhesive free and insert free. For child users, the position is to be determined but will include more of the genital mucosa.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved portable female urinal which has all of the advantages of the prior art urinals and none of the disadvantages.

It is another object of the present invention to provide a new and improved portable female urinal which may be easily and efficiently manufactured and marketed.

It is a further object of the present invention to provide a new and improved portable female urinal which is of durable and reliable constructions.

An even further object of the present invention is to provide a new and improved portable female urinal which is susceptible of a low cost of manufacture with regard to both materials and labor, and which accordingly is then susceptible of low prices of sale to the consuming public, thereby making such portable female urinal economically available to the buying public.

Lastly, it is an object of the present invention to provide a new and improved portable female urinal for facilitating inter-labial positioning and urine collecting, the positioning being within the labia minora allowing the collecting to be done in a sanitary, safe, discreet, convenient and economical manner.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated the preferred embodiment and alternate embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front elevational view of a portable female urinal system constructed in accordance with the principles of the present invention.

FIG. 1A is a front elevational view of a user with panties.

FIG. 1B is a perspective illustration of a container ready for positioning and use.

FIG. 2 shows the area of the female anatomy where the urinal of the present invention is received.

FIG. 3 is a cross sectional view taken along line 3-3 of FIG. 1.

FIG. 4 is a cross sectional view taken along line 4-4 of FIG. 3.

FIG. 5 is a plan view taken along line 5-5 of FIG. 1.

FIG. 6 is an exploded perspective view of the container and tube of FIG. 1.

FIG. 7 is an exploded perspective view of the container and tube of FIG. 6 but after use.

FIG. 8 is an exploded view similar to FIG. 6 but illustrating an alternate embodiment of the invention.

FIG. 9 is a side elevational view of a container constructed in accordance with another alternate embodiment of the invention.

FIG. 10 is a plan view of the FIG. 9 embodiment taken along line 10-10 of FIG. 9.

FIG. 11 is a front elevational view of the FIG. 9 embodiment taken along line 11-11 of FIG. 9.

FIG. 12 is a cross sectional view of the FIG. 9 embodiment taken along line 12-12 of FIG. 10 but illustrating the catheter positioned for use.

FIG. 13 is a cross sectional view taken along line 13-13 of FIG. 12.

FIG. 14 is a side elevational view of an alternate embodiment of the tube.

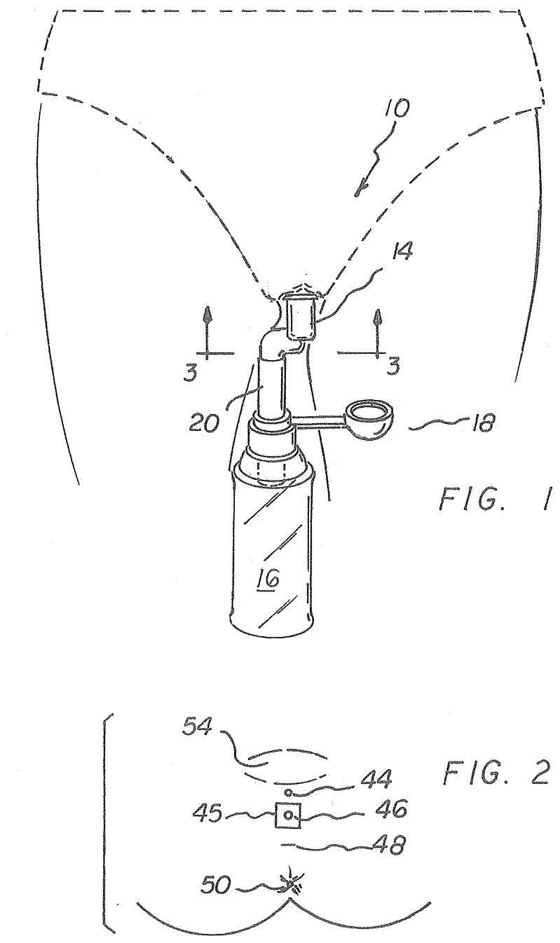

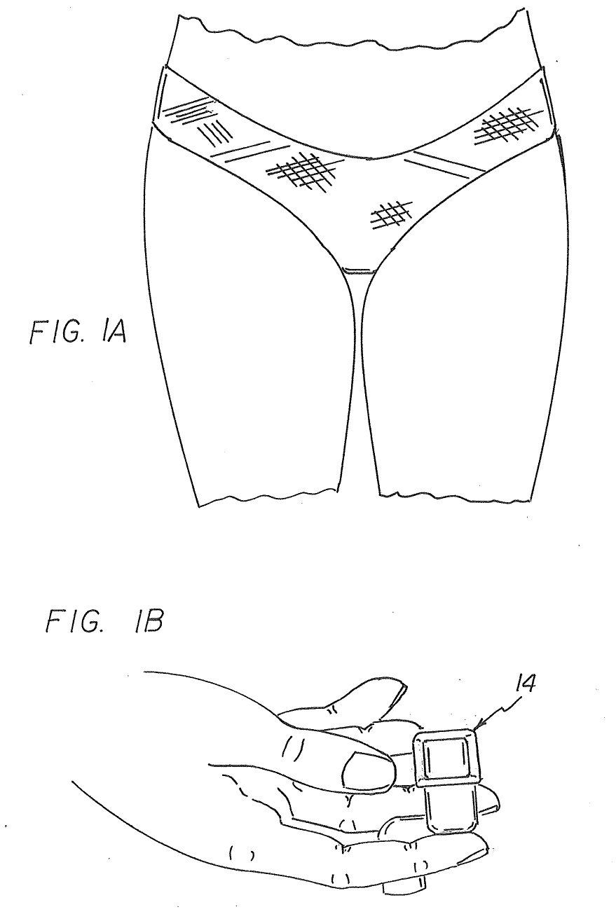

The same reference numerals refer to the same parts throughout the various Figures.

### DESCRIPTION OF THE PREFERRED EMBODIMENT

With reference now to the drawings, and in particular to FIG. 1 thereof, the preferred embodiment of the new and improved portable female urinal embodying the principles and concepts of the present invention and generally designated by the reference numeral 10 will be described.

In the preferred embodiment of the invention, a female portable urinal 10 is provided for facilitating inter-labial positioning and urine collecting. The positioning and collecting is done in a sanitary, safe, discreet, convenient and economical manner. The system includes an inter-labial collector 14 in a cylindrical configuration, a container 16, a container closure 18 and a tube 20 receiving the lower end of the container. The collector, referred to herein as a PEECO, initially receives the urine from the user. The container accepts the initially received urine from the collector for disposal or recycling or other usage. The tube couples the collector and the container. The tube 20 in the primary embodiment is linear. The tube 21 may, however, be arcuate or curved as illustrated in FIG. 14.

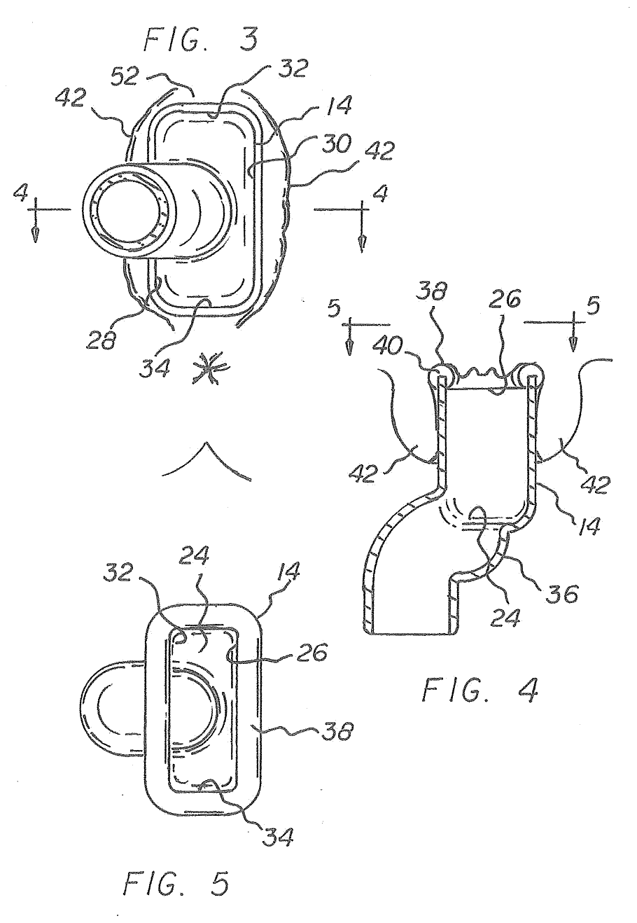

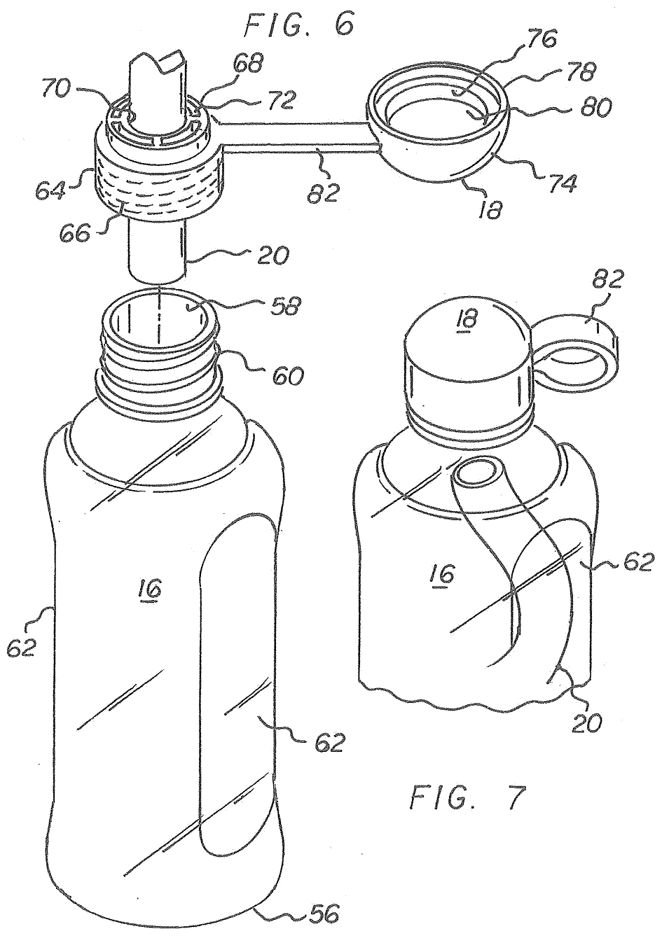

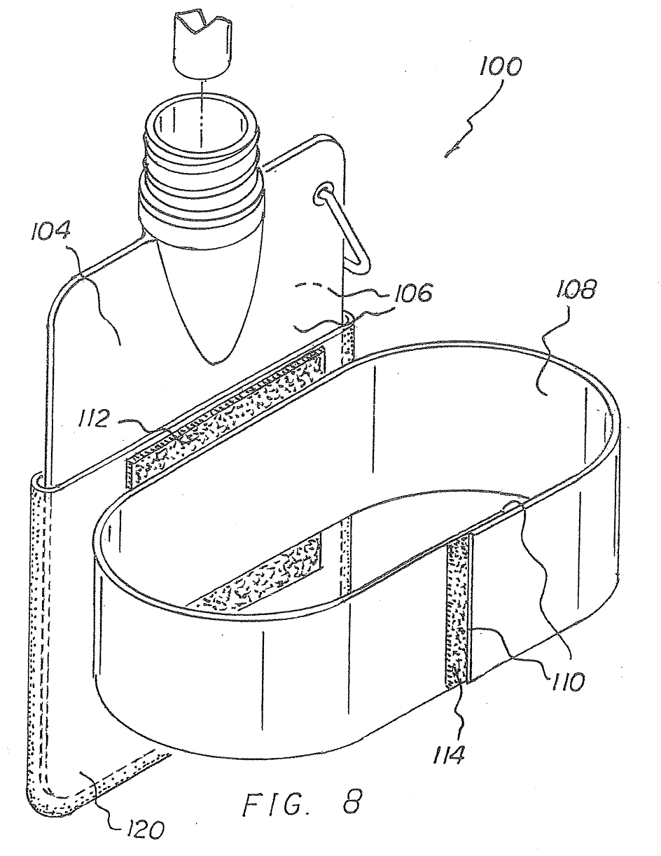

The inter-labial collector has a generally rectilinear configuration with a bottom 24. An opening is in the bottom for draining. The collector has an open top 26. Note FIG. 3 which illustrates the inter-labial collector from the bottom. FIG. 5 illustrates the inter-labial collector rom the top. FIG. 4 is a cross sectional view vertically through the center of the inter-labial collector.

Use of the system is as follows:

1) The user opens the labia minora in front of the anterior wall of the vagina using the non-dominant/left hand. This is best achieved in partial sitting position, with knees and hips flexed at about 50 degrees.

2) The PEECO is placed on the anterior wall of the vagina from a posterior or rear approach, using the dominant/right hand. Slide the PEECO forward into position in the vulvar vestibule, posterior to the clitoris with the labia minora open and PEECO over the urethral meatus.

3) User removes the dominant/right hand and the left/non-dominant hand begins to hold the PEECO from the front. After transfer of the PEECO from the rear or dominant to the front or non-dominant hand, the user stands closing labia minora over the sides of PEECO reducing the risk of leakage.

4) While holding the PEECO with the attached exit straw, using the non-dominant hand, a container is held in place by the dominant hand beneath the exit straw to receive urine channeled from the bladder.

5) With the non-dominant/left hand holding the PEECO anteriorly or from the front, and the dominant/right hand on the transparent container, the user voluntarily begins voiding until the container is as full as desired. Urine flow is voluntarily stopped and started to fill the container as needed and to empty the bladder periodically as needed.

The PEECO bottom and the open top are separated centrally by a depth of 4.0 centimeters plus or minus 25 percent. The inter-labial collector has a left side wall 28 and a parallel right side wall 30 separated by a width of 2.0 centimeters plus or minus 25 percent. The inter-labial collector has a front wall 32 and a rear wall 34 separated by a length of 4.0 centimeters plus or minus 25 percent. The inter-labial urinal has a bottom outlet continuous with an opening in the side of the cylinder 36 extending laterally from the bottom. The cylinder is configured to extend laterally, then downwardly at a 90 degree angle. Such configuration facilitates convenient positioning of the collector during use by the fingers of the user. The bottom and side walls and front and rear walls and cylinder are fabricated of a generally rigid plastic material with limited flexibility.

The side walls and front and rear walls form a peripheral edge 38 adjacent to the open top. A soft, pliable air-filled chamber 40 covers the peripheral edge. The air filled chamber has a diameter of 3.00 millimeter plus or minus 25 percent. The peripheral edge is adapted to be formed of any of a plurality of other soft, pliable materials such as a rolled edge or a foam elastomer. The inter-labial collector during use is laterally positionable and held inter-labially solely by a user's fingers, adhesive free and insert free between the labia minora 42 to cover a user's genitalia including clitoris 44, the vulvar vestibule 45, and the urethral orifice 46 forwardly of the vagina 48, with the collector in proximity to the front wall overlying the mucosa of the vulvar vestibule 50 and posterior to the rigid pubic symphysis 52 and with the chamber adjacent to the rear and side walls overlying soft tissue adjacent to the labia majora and anus 54.

The container has a closed base 56 and an upper opening 58 and a periphery. The upper opening has male threads 60. The container is adapted to be supported by a hand or thighs between the thighs of the user during use. The container is fabricated of a plastic material and adapted to receive urine from the inter-collector.

The container closure has a cylindrically-shaped component 64 with female threads 66 removably received on the male threads of the container. The cylindrically-shaped component has a raised central section 68 with a central passageway 70 and air exit vent holes 72. The container closure has a dome-shaped component 74. The dome-shaped component has an interior sealing ring 76 positionable on the raised central section. The dome-shape component also have an intermediate sealing ring 78 positionable on the cylindrically-shaped component adjacent to the raised central section. The dome-shaped component also having an exterior sealing ring 80 positionable on the cylindrically-shaped component remote from the raised central section. More or less sealing rings are adapted to be utilized as may be needed or desired. The container closure has a living hinge 82 coupling the cylindrically-shaped component and the dome-shaped component. The dome-shaped component is spaced from the cylindrically-shaped component prior to and during use. The dome-shaped component covers the cylindrically-shaped component after use. The container closure is fabricated of a plastic material.

The tube 20 has an upper end received on the outlet cylinder of the collector. The tube has a lower end positionable in the container. The tube has an intermediate extent positionable through the central passageway of the container closure during use. The entire tube is positioned within the container after use with the dome-shaped component positioned in a leakproof manner over the cylindrically-shaped component and the container.

Note is taken of FIG. 1A which shows a front elevational view of a user with panties. FIG. 1B is a perspective illustration of a container ready for positioning and use. Proper positioning of the container is simple after minimum education.

An alternate container 104 is fabricated of an elastomeric material. Such alternate container, prior to use, is configured generally flat with parallel inner and outer flat side faces 106 positionable between the user's thighs. In this manner, urine entering the alternate container will expand the side faces into a three dimensional shape. The alternate container includes a support pouch 120 in a rectilinear configuration with a closed bottom and closed sides and an open top removably receiving the container. The support pouch is constructed to include a mesh material for facilitating expansion during use. A strap 108 with free ends 110, hook and loop strips 114 on the strap adjacent to the free ends is adapted for positioning around a user's thighs. Hook and loop patches 112 on the support pouch and on the strap remote from the free ends to facilitate positioning of the alternate container between the user's thighs.

Hook and loop fasteners, such as Velcro-type fasteners, are disclosed as the preferred mechanisms for releasably coupling the various components of the system. Velcro is a trademark of Velcro Industries B.V. Limited Liability Company of Netherlands. It should be understood, however, that a wide variety of releasable fasteners such as hooks and eyes, snaps, buttons and loops, clasps and the like could readily be utilized.

In all of the embodiments, the urinal is laterally positionable to cover the user's genitalia including the vulvar vestibule and urethral orifice. The urinal has a front wall positionable overlying the mucosa posterior to the clitoris and the rigid pubic symphysis of the user. Also, the urinal has rear and side walls positionable overlying soft tissue adjacent to the labia majora.

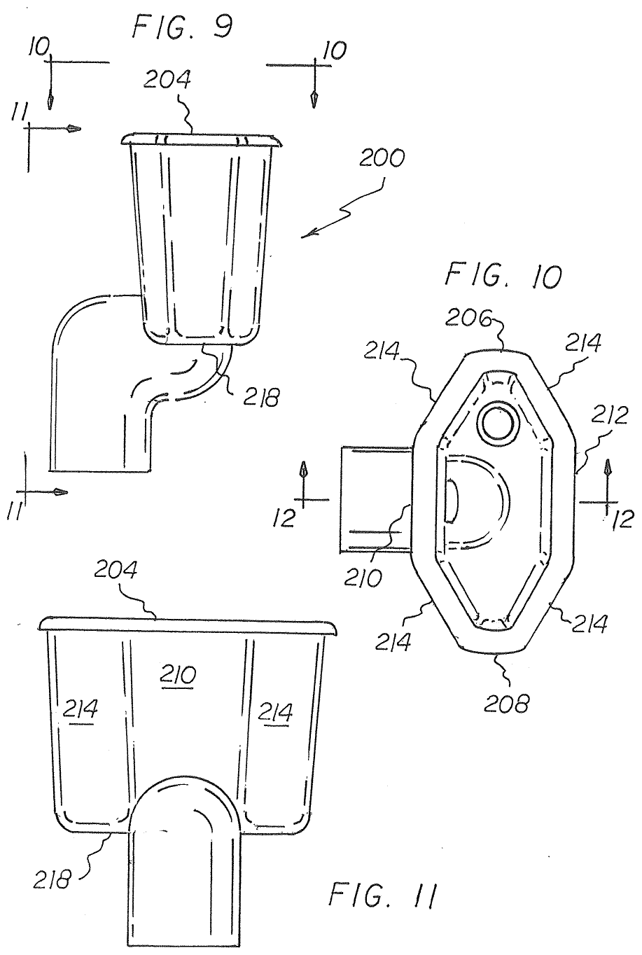

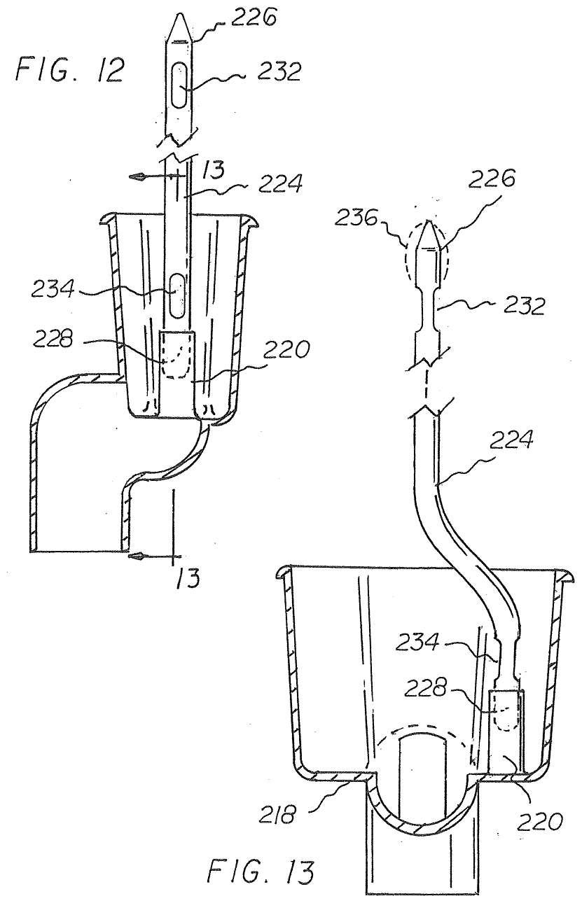

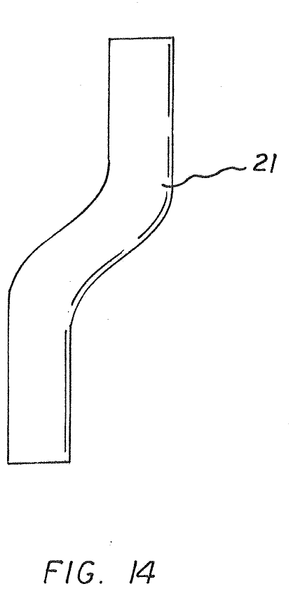

An additional alternate embodiment 200 of the invention is illustrated in FIGS. 9 through 13. In such embodiment, the container 204 is formed with an octagonal opening. Such opening has an upper edge 206 and a parallel lower edge 208 of a common length separated by a height of 2.0 centimeters plus or minus 25 percent. Such opening has a left side edge 210 and a right side edge 212 of a common length separated by a width of 1.0 centimeters plus or minus 25 percent. The left and right side edges have a common length of 1.0 centimeter plus or minus 25 percent. In addition, angled intermediate edges 214 are of a common length and are formed between the upper and lower and side edges. The octangular opening is thus in a diamond like configuration for greater comfort to the user. The side edges are longer than the angled intermediate edges, and the angled intermediate edges are longer than the upper and lower edges. Upper apertures 232 are for urine entrance from the user. Lower apertures 234 are for urine moving to the container.

It is noted that the container 200 includes a bottom 218 with a hollow stub 220. The hollow stub has a stub height, and the container has a container height. The stub height is between 25 percent and 50 percent of the container height. In association with the stub is a catheter 224. The catheter has an upper end 226 positionable in the user. The catheter has a lower end 228 inserted in the stub whereby the system is adapted for use by a user with need for intermittent catheterizing.

The catheter technique is as follows:

1) User assumes position of partial squat (as previously) which opens vulvar vestibule for voiding ease.

2) The finder doughnut around the catheter is used to located the urethral meatus. Once the urethral meatus is found, the catheter is advanced within the sterile sleeve into the urethra. The catheter size is slightly smaller than the doughnut hole permitting the advance without contamination by the meatal mucosa. The doughnut itself is about 1 millimeter in width as a circumference and has the sleeve attached to it. The finder doughnut is a total of about 7 millimeters in diameter. The hole is 5 millimeters in diameter and the catheter is a 14 French which is slightly smaller. The sleeve must be long enough to cover a catheter but with the 4.5 cm in length to extend from the rim of the PEECO to the urine in the bladder.

3) The sterile catheter is advanced through the sleeve approximately 3.0 cm or approximately ¾ of the 75 percent of the distance to the bladder from the vulvar vestibule.

4) The user resumes a standing position, transferring from the hand holding the catheter PEECO from the dominant to the non-dominant hand and in the standing position places the non-dominant/left hand on the PEECO from the front allowing the non-dominant hand to get a container for collection. The container for collection is placed at the end of the exit straw to channel bladder contents through the catheter and the PEECO into the container.

5) Advance the sterile catheter through the sleeve for the remaining distance to the bladder while watching the urine flow into the container.

6) Empty bottle as needed or obstruct flow as needed.

7) Remove catheter and discard it.

It should be understood that the container or multiple containers may be filled, sealed, and collected for disposal or use at a later time and/or location. Use of urine in the generation of electricity is the subject of current experimentation.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A portable urinal comprising: a collector
a collector having a bottom and an open top and a peripheral wall, the collector having an outlet opening, the bottom and the peripheral wall forming a chamber, the chamber having a front and rear and sides and a peripheral edge adjacent to the open top, the peripheral edge being positionable inter-labially between the labia minora to cover a user's urethral orifice, the peripheral wall including a generally rectangular front wall and a generally rectangular rear wall separated by a length, the peripheral walls including a generally rectangular left side wall and a generally rectangular parallel right side wall separated by a width of 2.0 centimeters plus or minus 25 percent for positioning laterally interior of a user's labia minora, the peripheral wall having linear edges in a rectangular configuration, with the chamber front positioned in the direction of soft tissue overlying the user's rigid pubic symphysis and with the chamber rear overlying soft tissue in proximity to the labia majora, vagina and anterior to the anus, the collector being held in place during use solely by the fingers of the user adhesive free and insert free.

2. A portable urinal comprising:
an inter-labial urinal having a bottom and an open top, the inter-labial urinal having a peripheral wall, the inter-labial urinal having an outlet cylinder, the peripheral wall formed with a peripheral edge at the open top, a covering at the peripheral edge, the inter-labial urinal having a left side wall and a right side wall separated by a width, the inter-labial urinal having a front wall and a rear wall separated by a length, the outlet cylinder extending downwardly from the inter-labial urinal, the bottom and side walls and front and rear walls forming a chamber and being fabricated of a generally rigid plastic material with limited flexibility, the side walls and front and rear walls forming the peripheral edge at the open top, the peripheral edge of the open top of the side walls of the inter-labial urinal being laterally positionable inter-labially between the labia majora to cover soft mucosal tissue of a user's genitalia surrounding the urethral orifice, within the labia majora, anterior to the anus, and extending anteriorly toward the soft tissue overlying the pubic symphysis, the peripheral walls including a generally rectangular front wall and a generally rectangular rear wall separated by a length, the peripheral walls including a generally rectangular left side wall and a generally rectangular parallel right side wall separated by a width of from 2.0 centimeters plus or minus 25 percent for positioning laterally interior of a user's labia majora and over vestibule, the peripheral walls having linear edges in a rectangular configuration, the peripheral edge of the open top of the side walls of the inter-labial urinal being held in place adhesive and insert free during use solely by the user;
a container having an upper opening and a periphery; and
a tube with an upper end received on the outlet cylinder of the inter-labial urinal, the tube having a lower end positionable in the container, the tube having an intermediate extent positionable through a central passageway of the container upper opening during use.

3. The system as set forth in claim 2 wherein the covering at the peripheral edge is a covering chosen from the class of coverings including an air-filled tube, a rolled edge and foam.

4. The system as set forth in claim 2 wherein the inter-labial urinal is fabricated of a plastic material.

5. The system as set forth in claim 2 wherein the container is fabricated of a plastic material.

6. The system as set forth in claim 2 wherein the container is fabricated of a thermochromic material to indicate recent usage.

7. The system as set forth in claim 2 wherein the upper opening of the container has threads and wherein the system further includes:
a container closure including a first component with female threads removably received on the male threads of the container, the first component having a central passageway and at least one air exit vent hole, the container closure having a second component positionable on the first component, the container closure having a living hinge coupling the first component and the second component, the container closure being fabricated of a plastic material.

8. The system as set forth in claim 7 wherein the entire tube is positioned within the container after use with the second component positioned in a leakproof manner over the first component and the container.

9. The system as set forth in claim 2 wherein the bottom of the inter-labial urinal is planar.

10. The system as set forth in claim 2 wherein the container (16) is fabricated of an elastomer with limited flexibility and includes parallel flat side faces (66) to facilitate positioning of the container in anticipation of use and during use.

11. The system as set forth in claim 2 wherein the container (104) is fabricated of a flexible elastomer, chosen from the class of flexible elastomers including plastic and rubber, natural and synthetic, and blends thereof, the container, prior to use being flat with parallel flat side faces (106) positionable between the user's thighs wherein urine entering the container will expand the side faces into a three dimensional shape, the container including a strap (108) with free ends (110), the container including releasable fastener patches (112) on the strap and on the side faces adapted to removably secure the strap to one side of the container, the strap has free ends with releasable fastener strips (114) for coupling the strap around a user's thigh to facilitate positioning of the container in anticipation of use and during use.

12. The system (200) as set forth in claim 2 wherein the opening (204) is an octagonal opening in shape with an upper edge (206) and a parallel lower edge (208) of a common length separated by a height, the octagonal opening has a left side edge (210) and a right side edge (212) of a common length separated by a width and four angled intermediate edges (214) of a common length formed between the upper and lower and side edges, the side edges being longer than the angled intermediate edges, and the angled intermediate edges being longer than the upper and lower edges.

13. The system (200} as set forth in claim 2 wherein the container includes a bottom (218) with a hollow stub (220) with a stub height, and the container has a container height, the stub height being between 25 percent and 50 percent of the container height, the system including a catheter (224), the catheter having an upper end (226) positionable in the user, the catheter having a lower end (228) inserted in the stub whereby the system is adapted for use by a users with a catheterizing issue, the catheter having upper apertures (232) for urine entrance from the user, the catheter having lower apertures (234) for urine moving to the container.

14. A female portable urinal (10) for facilitating inter-labial positioning and urine collecting, the positioning and collecting being done in a sanitary, safe, discreet, convenient and economical manner, the system comprising, in combination:
an inter-labial collector (14), a container (16), a container closure (18) and a tube (20) functioning as an exit straw;
the inter-labial collector having a generally rectilinear configuration with a bottom (24), an opening in the bottom to the drain and an open top (26), the bottom and the open top being separated centrally by a depth of 4.0 centimeters plus or minus 25 percent, the inter-labial collector having a left side wall (28) and a parallel right side wall (30) separated by a width of 2.0 centimeters plus or minus 25 percent, the inter-labial collector having a front wall (32) and a rear wall (34) separated by a length of 4.0 centimeters plus or minus 25 percent, the inter-labial urinal having an outlet cylinder (36) extending from the bottom, the bottom and side walls and front and rear walls being fabricated of a generally rigid plastic material with limited flexibility, the side walls and front and rear walls forming a peripheral edge (38) adjacent to the open top, a soft pliable air-filled chamber (40) covering the peripheral edge, the air filled chamber having a diameter of 3.00 millimeter plus or minus 25 percent, the inter-labial collector during use being laterally positionable and held inter-labially solely by a user's fingers, adhesive free and insert free between the labia majora covering soft tissue of a user's genitalia surrounding the urethral orifice, within the labia majora, anterior to the anus, and extending anteriorly toward the soft tissue overlying the pubic symphysis;
the container having a closed base (56) and an upper opening (58) and a periphery, the upper opening having male threads (60), the container adapted to be supported between the thighs of the user during use, the container being fabricated of a plastic material and adapted to receive urine from the inter-collector;
the container closure having a cylindrically-shaped component (64) with female threads (66) removably received on the male threads of the container, the cylindrically-shaped component having a raised central section (68) with a central passageway (70) and air exit vent holes (72), the container closure having a dome-shaped component (74), the dome-shaped component having an interior sealing ring (76) positionable on the raised central section, the dome-shape component also have an intermediate sealing ring (78) positionable on the cylindrically-shaped component adjacent to the raised central section, the dome-shaped component also having an exterior sealing ring (80) positionable on the cylindrically-shaped component remote from the raised central section, the container closure having a living hinge (82) coupling the cylindrically-shaped component and the dome-shaped component, the dome-shaped component being spaced from the cylindrically-shaped component prior to and during use, the dome-shaped component covering the cylindrically-shaped component after use, the container closure being fabricated of a plastic material;
the tube (20) having an upper end received on the outlet cylinder of the collector, the tube having a lower end positionable in the container, the tube having an intermediate extent positionable through the central passageway of the container closure during use, the entire tube positioned within the container after use with the dome-shaped component positioned in a leakproof manner over the cylindrically-shaped component and the container; and
a container (104), fabricated of an elastomeric material and configured, prior to use, generally flat with parallel inner and outer flat side faces (106) positionable between the user's thighs wherein urine entering the container will expand the side faces into a three dimensional shape, the container including a support pouch (120) in a rectilinear configuration with a closed bottom and closed sides and an open top removably receiving the container, the support pouch being constructed to include a mesh material for facilitating expansion during use, a strap (108) with free ends (110), hook and loop strips (114) on the strap adjacent to the free ends adapted for positioning around a user's thigh, hook and loop patches (112) on the support pouch and on the strap remote from the free ends to facilitate positioning of the container between the user's thighs.
